(19) United States
(12) Patent Application Publication
(10) Pub. No.: US 2023/0414093 A1
(43) Pub. Date: Dec. 28, 2023

(54) ENHANCED VISION SCREENING USING EXTERNAL MEDIA

(71) Applicant: Welch Allyn, Inc., Skaneateles Falls, NY (US)

(72) Inventors: Carlos Suarez, Syracuse, NY (US); John A. Lane, Venice, FL (US); Timothy R. Fitch, Skaneateles, NY (US)

(21) Appl. No.: 18/213,634

(22) Filed: Jun. 23, 2023

Related U.S. Application Data
(60) Provisional application No. 63/355,050, filed on Jun. 23, 2022.

(51) Int. Cl. A61B 3/032; A61B 3/10

(52) U.S. Cl. CPC A61B 3/032; A61B 3/102

(57) ABSTRACT

An example method performed by a vision screening device includes receiving, from an external medium, a signal and identifying a vision test associated with the external medium based on the signal. The vision screening device receives feedback characterizing the vision test from a subject. In addition, the vision screening device determines whether the subject is suspected to have an ocular condition based on the feedback based on the vision test.

### CROSS-REFERENCE TO RELATED APPLICATIONS

This application claims priority to and is a non-provisional application of U.S. Provisional Patent Application No. 63/355,050 filed on Jun. 23, 2022, the entire contents of which are incorporated herein by reference.

### TECHNICAL FIELD

This application relates generally to vision screeners for performing vision tests using external media.

### BACKGROUND

Mass vision screening events are utilized to assess the vision of many children in a short amount of time. Users operating on-site vision screening devices can assess children directly. For example, a user without specialized training can operate a vision screening device to perform an autorefraction assessment on multiple children at a screening event. These tests can be performed without feedback from the children being tested. For example, the vision screening device may project an infrared pattern on the eye of a child, and identify whether the child should follow up for a formal eye exam, by evaluating the reflection of the pattern on the eye.

Some vision tests, however, require feedback. For example, a color vision test can be utilized to assess whether a child subjectively ascertains a particular color pattern. However, existing vision screening devices are not equipped to receive feedback from children. Some children may be illiterate or minimally literate, and would struggle to provide feedback about vision tests using conventional computer-based user interfaces. Currently, these feedback-dependent tests are administered to children manually by trained practitioners, which is not conducive to mass screening.

In addition, some vision tests can be offered in a variety of formats. For example, color vision tests can be Ishihara tests or Color Vision Test Made Easy (CVTME) examinations. Furthermore, different jurisdictions may have policies that implement different vision tests. For example, one state may require children to be screened with an Ishihara test, whereas another state may require children to be screened with a CVTME test.

### SUMMARY

Various implementations of the present disclosure relate to techniques for vision screening devices that can assess the vision tests on subjects using external media. These devices may be suitable for assessing conditions of multiple subjects in mass screening events, such as screening events conducted in schools.

In various cases, a vision test is visually output by an external medium, such as a card, a poster, or a computing device. A vision screening device may identify the vision test output by the external medium. For example, the external medium may display and/or transmit a code indicative of the vision test to the vision screening device. The vision screening device may identify feedback characterizing the vision test from a subject. In some cases, the subject directly inputs the feedback, or a user may input the feedback. The vision screening device, in various implementations, may identify whether the subject is suspected and/or expected to have one or more ocular conditions by analyzing the feedback in view of the identified vision test.

According to some examples, a single vision screening device can assess conditions of subjects using a large variety of different vision tests. In some cases, the vision screening device is compatible with external media configured to output different vision tests to subjects. The vision screening device may identify a particular vision test being output to a particular subject by an external medium in order to evaluate a condition of the subject. By incorporating vision tests output by external media, the vision screening device may evaluate subjects based on feedback characterizing a wide variety of vision tests.

In some cases, the vision screening devices can facilitate reception of feedback characterizing the vision tests from subjects (e.g., children) who may be unable to operate complex user interfaces. According to some examples, the feedback can be input by a user who is different than the subject being evaluated. In some cases, the subject can input the feedback by tracing a shape on a touchscreen, speaking the feedback, selecting graphic icons, or other user-friendly methods that are achievable by children.

Various implementations of the present disclosure are directed to technological improvements in the field of vision screening devices. Existing vision screening devices, designed for mass screening, are unable to provide vision tests (e.g., color vision tests, visual acuity tests, etc.) that are evaluated based on subject feedback. Accordingly, these tests are often excluded from mass screening events. By incorporating the use of external media, various example vision screening devices described herein can facilitate administering feedback-oriented vision tests to many subjects in a short amount of time.

### DESCRIPTION OF THE FIGURES

The following figures, which form a part of this disclosure, are illustrative of described technology and are not meant to limit the scope of the claims in any manner.

FIG. 1 illustrates an example environment for performing vision tests using external media.

FIG. 2 illustrates example signaling for vision screening using external media.

FIG. 3 illustrates an example vision screening device with an external medium for administering a vision test to a subject.

FIGS. 4A and 4B illustrate another example vision screening device with an external medium for administering a vision test to a subject.

FIG. 5 illustrates a further example vision screening device with an external medium for administering a vision test to a subject.

FIG. 6 illustrates an additional example vision screening device with an external medium for administering a vision test to a subject.

FIG. 7 illustrates yet another example vision screening device with an external medium for administering a vision test to a subject.

FIG. 8 illustrates an example vision screening device in which a removeable external medium is mounted on the vision screening device.

FIG. 9 illustrates another example vision screening device with removeable external media that can be selectively attached to the vision screening device.

FIGS. 10A to 10D illustrate examples of feedback devices including external media for administering a vision test to a subject.

FIG. 11 illustrates a vision screening device packaged with cards that serve as external media.

FIGS. 12A to 12C illustrate an example workflow for administering a vision test to a subject using a tablet as an external medium and feedback device.

FIGS. 13A and 13B illustrate an example workflow for vision screening using a vision screening device that includes a first screen and a second screen.

FIGS. 14A and 14B illustrate a feedback device configured to receive feedback directly from a subject.

FIGS. 15A to 15C illustrate a workflow for vision screening in which a handheld card is used as an external medium and a tablet is used as a feedback device.

FIGS. 16A to 16C illustrate a workflow for vision screening in which a poster is used as an external medium and a tablet is used as a feedback device.

FIGS. 17A and 17B illustrate a workflow for vision screening in which a laptop is used as an external medium and a tablet is used as a feedback device.

FIG. 18 illustrates an example process for vision screening using external media.

FIG. 19 illustrates at least one example device configured to enable and/or perform the some or all of the functionality discussed herein.

### DETAILED DESCRIPTION

Various implementations of the present disclosure will be described in detail with reference to the drawings, wherein like reference numerals present like parts and assemblies throughout the several views. Additionally, any samples set forth in this specification are not intended to be limiting and merely set forth some of the many possible implementations.

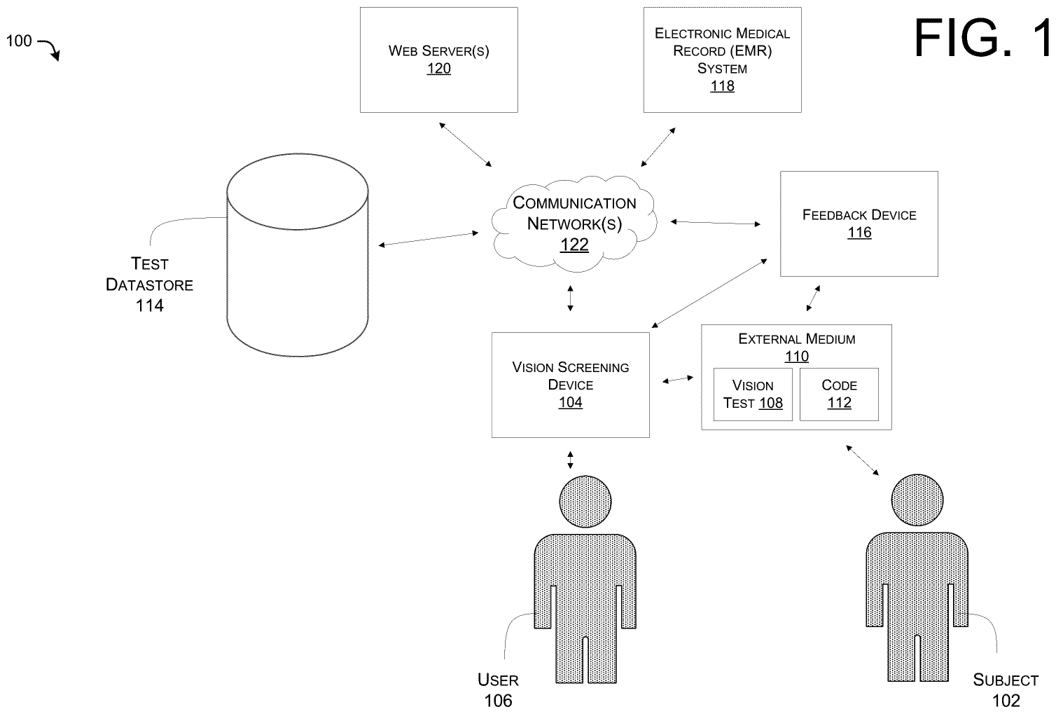

FIG. 1 illustrates an example environment 100 for performing vision tests using external media. In various implementations, at least one eye of a subject 102 is screened for at least one ocular condition. As used herein, the terms “ocular condition,” “ophthalmic condition,” “condition,” and their equivalents, can refer to a pathologic state of an individual that is associated with a state of at least one eye of the individual. Some ocular conditions, for example, are pathological conditions of the eye itself, such as amblyopia, myopia, hyperopia, astigmatism, cataract, retinopathy, color vision deficiency, macular degeneration, and so on. Some ocular conditions are pathological conditions of other areas of the body, but can be identified based on the appearance and/or performance of the eye. Other examples of ocular conditions include concussion, learning disorders (e.g., dyslexia), some cancers, and so on.

A vision screening device 104 is configured to determine whether the subject 102 is suspected to have one or more ocular conditions. As used herein, a subject may be “suspected to have,” and/or “likely to have” a condition if at least one parameter associated with the subject is outside of a range associated with a particular screening exam. For example, the vision screening device 104 may not specifically diagnose the subject 102 with an ocular condition, but may determine whether the subject 102 should be evaluated by a trained care provider (e.g., an optometrist or ophthalmologist) for the ocular condition. That is, the vision screening device 104 may be a tool for determining whether a follow-up examination is indicated for the subject 102. The vision screening device 104 may be operated by a user 106. As shown, the user 106 is different than the subject 102, but implementations are not so limited. According to various implementations, the user 106 screens multiple subjects including the subject 102 for one or more ocular conditions in a mass screening event. For example, the subjects could be children at a school, residents of a nursing home, or other groups who are screened in a relatively short amount of time.

In various implementations, the vision screening device 104 identifies the performance of the subject 102 on a vision test 108 output by an external medium 110. As used herein, the term “vision test,” and its equivalents, can refer to displayed information that can be used to assess the vision of an individual. For example, the vision test 108 may include a color deficiency test, which can also be referred to as a “color blindness” test. Examples of color deficiency tests include Ishihara tests and CVTME tests. The vision test 108 may include one or more pictures that display a symbol (e.g., a number) in at least one first color and at least one second color as a background to the symbol. If the subject 102 has sufficient color sensitivity, the subject may see the symbol. If the subject 102 is color deficient, the subject 102 may be unable to discern the symbol.

In some cases, the vision test 108 includes a visual acuity test. According to some implementations, a visual acuity test displays symbols of with different sizes. The visual acuity of the subject 102 is determined based on the sizes at which the subject 102 can visually recognize one or more of the symbols. There are multiple types of visual acuity tests, such as near vision tests and distance vision tests. Near vision tests display the symbols at a relatively close distance from the eye of the subject 102, such as 35 centimeters (cm). The results of a near vision test are indicative of whether the subject 102 is nearsighted. Distance vision tests display the symbols at a relatively long distance from the eye of the subject 102, such as 6 meters (m). The results of a distance vision test are indicative of whether the subject is farsighted.

In various examples, the vision test 108 includes a reading speed test. For example, the vision test 108 displays multiple words. The speed at which the subject 102 reads the words corresponds to the reading speed of the subject 102. In some instances, the vision test 108 includes a reading comprehension test. The vision test 108 may display a passage of words. Upon reading the passage, the subject 102 may indicate what the passage discusses, thereby demonstrating whether the subject 102 adequately understands the passage. Reading speed tests and reading comprehension tests may be used to assess whether the subject 102 has a learning disability or other condition.

According to various implementations, the vision test 108 includes a concussion test. For instance, the vision test 108 may include a test described in U.S. Pat. No. 10,506,165, which is incorporated by reference herein in its entirety. For instance, the vision test 108 may include one or more symbols that the subject 102 focuses on visually. The vision screening device 104 may capture one or more images of the eyes of the subject 102 while the subject is focusing on the vision test 108. In some cases, the vision screening device 104 determines a pupil size of the subject 102 based on the image(s) and determines whether the subject 102 is predicted to have a concussion based on a pupil size of the subject 102.

In some cases, the vision test 108 is gamified for the subject 102. For example, the external medium 110 may display a shape (e.g., a butterfly) that moves along the external medium 110. The subject 102 may play a game by inputting feedback based on the position of the shape. For example, the subject 102 may “capture” a virtual butterfly displayed by the external medium 110 by controlling an input device (e.g., a touchscreen), and based on the feedback, the vision screening device 104 may evaluate the vision of the subject 102.

In various examples, the vision test 108 is displayed by the external medium 110. As used herein, the term “external medium,” and its equivalents, can refer to a device and/or object that is separate from a device used to identify the results of a vision test (e.g., the vision screening device 104). In some cases, the external medium 110 includes a substrate (e.g., a passive object), such as a projection screen reflecting a projection of the vision test 108, a poster displaying the vision test 108, a card displaying the vision test 108, or some other printed substrate displaying the vision test 108. As used herein, the term “substrate,” and its equivalents, can refer to a solid or semisolid material that can absorb and/or reflect light. In various examples, the external medium 110 includes an active device, such as a tablet computer or smartphone that displays the vision test 108 on a touchscreen, a smart TV that displays the vision test 108, a virtual reality (VR) headset that displays the vision test 108, an augmented reality device that displays the vision test 108, or some other computing device that displays the vision test 108 on a screen.

According to various implementations, the vision screening device 104 may be configured to assess the results of multiple different vision tests including the vision test 108. To identify the vision test 108 among the multiple vision tests, the vision screening device 104 may identify a code 112 that is associated with the vision test 108. As used herein, the term “code,” and its equivalents, can refer to one or more symbols that indicate the identity of a vision test. In some examples, the code 112 is displayed on the external medium 110 with the vision test 108. For example, the vision screening device 104 includes a camera that captures an image of the code 112 on the external medium 110. As used herein, the term “image,” and its equivalents, can refer to a set of data including multiple pixels and/or voxels that respectively represent regions of a real-world scene. A two-dimensional (2D) image is represented by an array of pixels. A three-dimensional (3D) image is represented by an array of voxels. An individual pixel and/or voxel in an image is defined according to at least one value representing an amount and/or frequency of light emitted by the corresponding region in the real-world scene.

In some implementations, a signal indicative of the code 112 is transmitted from the external medium 110 to the vision screening device 104. For instance, the vision screening device 104 includes a transceiver that receives a signal (e.g., a wireless signal) indicative of the code 112 from the external medium 110. In some examples, the vision screening device 104 and/or the external medium 110 may be connected, such as via a Bluetooth connection and/or a Nearfield Communication (NFC). For example, the vision screening device 104 may be paired to the external medium 110, or vice versa, such that the two devices may communicate with and send data to and from one another.

The code 112 may be uniquely associated with the vision test 108 displayed by the external medium 110, such that no other vision test is displayed with the code 112. In some cases, the code 112 is a barcode, such as a QR code. In various implementations, the code 112 is indicative of a string of one or more letters or numbers that are associated with the vision test 108. In various implementations, the vision screening 104 identifies the vision test 108 by identifying an entry in a test datastore 114 that includes the code 112. For example, the test datastore 114 includes a database and/or lookup table indexed by codes associated with respective vision tests. By finding the entry of the database with the code 112, the vision screening device 104 may identify the vision test 108. In some cases, the test datastore 114 is part of the vision screening device 104. In some examples, the test datastore 114 is hosted in a device that is external to the vision screening device 104.

The subject 102 may view the vision test 108 and produce feedback based on the vision test 108. As used herein, the term “feedback,” and its equivalents, can refer to data representing an individual's performance on a vision test. The feedback, for example, is detected by a feedback device 116. In some implementations, the feedback device 116 is part of the vision screening device 104 and/or the external medium 110. In various cases, the feedback device 116 includes a sensor configured to detect the feedback from the subject 102. In some examples, the feedback device 116 may be in communication with the vision screening device 104 and/or the external medium 110, such that the feedback device 116 may send data indicative of the feedback from the subject 102 to the screening device 104 and/or the external medium 110. The feedback device 116 may be in communication with the vision screening device 104 and/or the external medium 110 via a Bluetooth connection and/or a NFC connection, to name a few examples. In some examples, the feedback from the subject 116 may be send upon a determination, from the feedback device 116, that the vision screening is complete. For example, based on the feedback received from the subject 102, the feedback device 116 may determine that the test has concluded. Additionally, or alternatively, the feedback device 116 may receive an input, such as from the subject 102 and/or the user 106, of a conclusion of the test. In other examples, the feedback device 116 may send the results continuously, as they are received by the feedback device 116.

Various types of feedback can be detected by the feedback device 116. In some implementations, the feedback device 116 includes one or more touch sensors incorporated with the external medium 110. The feedback may be a touch of the subject 102 on at least a portion of the external medium 110. For example, the subject 102 may trace a symbol of the vision test 108, which is detected by the touch sensor(s) of the feedback device 116. In some cases, the subject 102 may touch an icon displayed on the external medium 110 that is detected by the feedback device 116 as the feedback from the vision test 108.

In various examples, the feedback device 116 includes one or more cameras that visually detect the feedback from the subject 102. For example, the vision test 108 may be a reading speed test and the camera(s) capture images of an eye of the subject 102 as the subject is reading the passage. The feedback may be the change in the gaze angle of the subject 102 over time.

In some cases, the feedback device 116 includes other types of input devices that can detect feedback directly from the subject 102. For example, the feedback device 116 may include a microphone that detects the voice of the subject 102 that serves as the feedback about the vision test 108. In some examples, the feedback device 116 includes physical buttons, a keyboard, or any other device configured to detect an input signal indicative of the feedback from the subject 102.

According to some examples, the user 106 inputs the feedback from the subject 102 into the feedback device 116. For example, the subject 102 may audibly report the feedback to the user 106, who may manually input the feedback into the feedback device 116 using a button, keyboard, touch screen, or other input device.

The feedback device 116 may provide the feedback to the vision screening device 104. In various implementations, the vision screening device 104 may determine whether the subject 102 is suspected to have an ocular condition by analyzing the feedback in view of the vision test 108. In some cases, the entry in the test datastore 114 indicating the vision test 108 may further include a key associated with the vision test 108. For instance, if the vision test 108 is an Ishihara color deficiency test, the key may be the identity of the symbol that is displayed in the vision test 108. The feedback device 116 may compare the feedback to the key. In other words, the feedback device may, based on receiving the feedback, compare the feedback to the key to determine one or more discrepancies between the feedback and the key, wherein one or more discrepancies may indicate the suspect is suspected to have an ocular condition. In some examples, a greater number of discrepancies may indicate a higher likelihood of an ocular condition, whereas a lower number of discrepancies may indicate a lower likelihood of an ocular condition. In some examples, the number of discrepancies may be over a threshold number of discrepancies, which may indicate that the subject 102 does have an ocular condition. In some examples, the vision screening device 108 may determine a type of ocular condition a subject 102 is suspect to have or has. For example, the feedback received from the subject 102 may correspond to various ocular conditions. In other words, tracing an object but failing to determine a correct color of the object may indicate that the subject 102 has adequate vision, but is colorblind. In some examples, determining that the subject is suspected to have or has an ocular condition (such as, for example, comparing the feedback to the key) is done manually by the user 104. However, in other examples, this may be done automatically by one or more algorithms of the vision screening deice 108. For example, the vision screening device 108 may trained to compare the feedback to the key to identify one or more discrepancies. Based at least in part on factors such as the type of discrepancies and/or number of discrepancies, for example, the feedback device 108 may output a likelihood that a subject 102 has a ocular condition, and/or the ocular condition(s) the subject 102 is likely to have.

In some implementations, the key is defined as a shape that is within a threshold distance (e.g., 1 centimeter) of the symbol. The feedback device 116 may determine whether the subject 102 traces a shape that is within the key. In some implementations, the subject 102 traces the symbol on a touchscreen, and the feedback may be highlighted on the touchscreen as the subject 102 is tracing the symbol. In some implementations, the subject 102 traces the symbol with a writing instrument (e.g., a marker, pen, or pencil) on a paper substrate. The highlighted and/or written feedback may be viewed manually. If the feedback matches the key, then the vision screening device 104 may determine that the subject 102 has passed the vision test 108. If the feedback is different than the key, then the vision screening device 104 may determine that the subject 102 has not passed the vision test 108. In various implementations, the vision screening device 104 may determine that the subject 102 is suspected to have an ocular condition based on determining that the subject 102 has not passed the vision test 108.

In some implementations, the key indicates a threshold that the vision screening device 104 compares to the feedback. For example, if the vision test 108 is a reading speed test, and the feedback represents a reading speed of the subject 102, the vision screening device 104 may compare the reading speed of the subject 102 to a threshold speed in order to determine whether the subject 102 is at an appropriate reading level or is suspected of having a learning disability.

The vision screening device 104 may perform additional tests on the subject 102 that are independent of the external medium. In various implementations, the vision screening device 104 performs an automated autorefraction assessment on the subject 102. For example, the vision screening device 104 may include at least one light source configured to project an infrared pattern on an eye of the subject 102. As used herein, the term “light source,” and its equivalents, can refer to an element configured to output light, such as a light emitting diode (LED) or a halogen bulb.

The vision screening device 104, in some instances, further includes at least one camera configured to capture an image of a reflection of the pattern from the eye of the subject 102. The vision screening device 104 may determine a condition of the subject 102 based on the reflection of the pattern. For example, the vision screening device 104 may determine that the subject has myopia, hyperopia, astigmatism, or a combination thereof, based on the reflection of the pattern. In some cases, the vision screening device 104 is or includes a specialized device, such as the Welch Allyn Spot Vision Screener by Hill-Rom Services, Inc. of Chicago, IL. In some cases, the vision screening device 104 performs a red reflex examination on the subject 102.

According to various implementations, the vision screening device 104 may output and/or store a result of the vision test 108 or the result of any other vision test identified by the vision screening device 104. The result, for example, is an indication of the feedback, a discrepancy between the feedback and the key, whether the subject 102 is suspected to have the ocular condition, or a combination thereof. In some implementations, the vision screening device 104 outputs the result to the user 106. For example, the vision screening device 104 may display the result on a screen and/or audibly output the result using a speaker. In some cases, the visions screening device 104 stores the result (e.g., with an indication of the identity of the subject 102).

In some cases, the vision screening device 104 determines an identity of the subject 102. For instance, the user 106 may input a code, name, or other identifier associated with the subject 102 into the vision screening device. The vision screening device 104 may generate and/or store the result with the identifier of the subject 102.

The vision screening device 104 may be communicatively coupled to an electronic medical record (EMR) system 118. In some cases, the vision screening device 104 transmits the result (and the identifier of the subject 102) to the EMR system 118. The EMR system 118 may include one or more servers storing EMRs of multiple individuals including the subject 102. As used herein, the terms “electronic medical record,” “EMR,” “electronic health record,” and their equivalents, can refer to a data indicating previous or current medical conditions, diagnostic tests, or treatments of a patient. The EMRs may also be accessible via computing devices operated by care providers. In some cases, data stored in the EMR of a subject is accessible to a user via an application operating on a computing device. For instance, the stored data may indicate demographics of a subject, parameters of the subject, vital signs of the subject, notes from one or more medical appointments attended by the subject, medications prescribed or administered to the subject, therapies (e.g., surgeries, outpatient procedures, etc.) administered to the subject, results of diagnostic tests performed on the subject, subject identifying information (e.g., a name, birthdate, etc.), or any combination thereof. In various implementations, the EMR system 118 stores the feedback and/or result in an EMR associated with the subject 102.

In some examples, the vision screening device 104 transmits the result to one or more web servers 120. In various implementations, the web server(s) 120 may store indications of the result. In addition, the web server(s) 120 may output a website to an external computing device (not illustrated) indicating the result. In some cases, the external computing device may be operated by a parent of the subject 102, such that the parent may view the indication of the result by accessing the website. In some implementations, the web server(s) 120 further stores additional information about the vision test 108 and/or recommended follow-up care for the subject 102. For instance, based on the result, the website may indicate that the subject 102 should be seen by an optometrist and/or ophthalmologist for follow-up care.

Various elements of the environment 100 communicate via one or more communication networks 122. The communication network(s) 122 include wired (e.g., electrical or optical) and/or wireless (e.g., radio access, BLUETOOTH, WI-FI, or near-field communication (NFC)) networks. The communication network(s) 122 may forward data in the form of data packets and/or segments between various endpoints, such as computing devices, medical devices, servers, and other networked devices in the environment 100.

Although not specifically illustrated in FIG. 1, the vision screening device 104, external medium 110, and feedback device 116 can be utilized to assess the vision of multiple subjects in a mass screening event. For example, 10, 100, or 1,000 subjects may be efficiently tested over the course of one or more days. Using the techniques described herein, the user 106 may operate the vision screening device 104 in order to assess the vision of the multiple subjects using the vision test 108 output by the external medium 110. In some cases, multiple external media outputting different vision tests can be utilized to assess the subjects using different vision tests. In some implementations, the same external medium 110 can output different vision tests to the subjects.

In particular examples, the vision screening device 104 is configured to assess the performance of the subject 102 on the vision test 108, which is output by the external medium 110. Using various implementations described herein, the vision screening device 104 is adapted to efficiently screen numerous subjects (including the subject 102) in a mass screening event, even in cases where the user 106 is not a trained clinician and/or when the subjects are children.

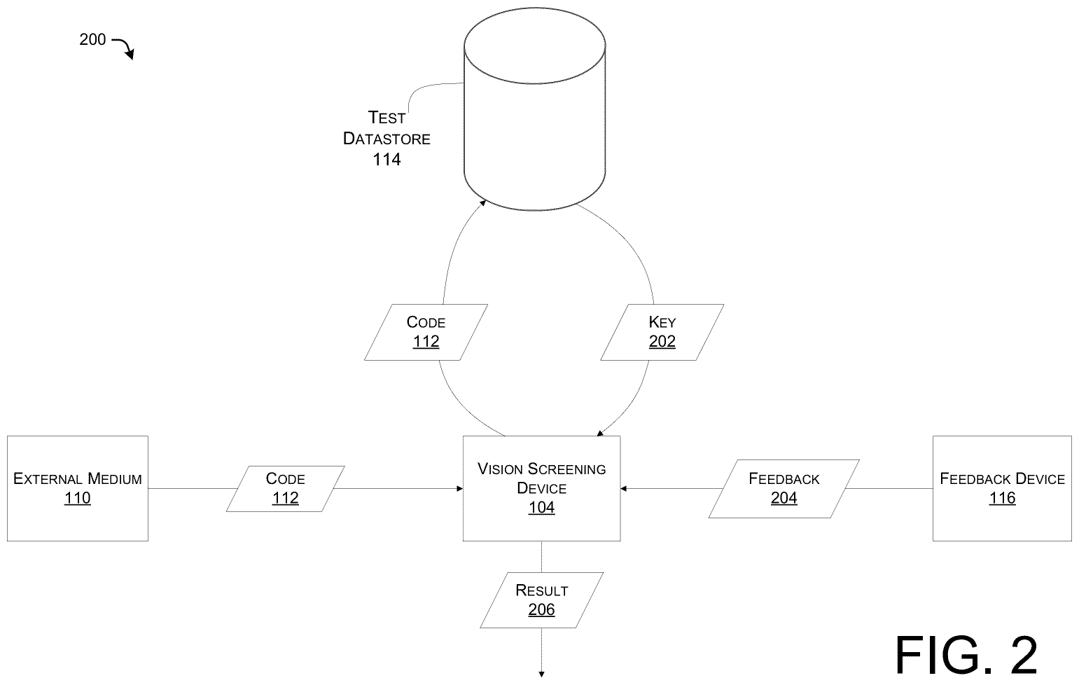

FIG. 2 illustrates example signaling 200 for vision screening using external media. The signaling 200 is between the vision screening device 104, the external medium 110, the test datastore 114, and the feedback device 116 described above with reference to FIG. 1. In various implementations, at least one of the external medium 110, the test datastore 114, or the feedback device 116 is a component of the vision screening device 104. The signaling 200 illustrated in FIG. 2 also utilizes the code 112 described above with reference to FIG. 1.

According to various examples, the vision screening device 104 facilitates vision testing of a subject. The external medium 110 may display or otherwise output a vision test to the subject. In some cases, the external medium 110 further outputs the code 112 to the vision screening device 104. The code 112, for example, is uniquely associated with the vision test that is output by the external medium 110.

The vision screening device 104 may identify the vision test using the code 112. For instance, the vision screening device 104 may identify an entry in the test datastore 114 that includes the code 112. In various cases, the entry includes a key 202 that is associated with the vision test. The vision screening device 104 may retrieve and/or receive the key 202 from the entry of the test datastore 114.

In various implementations, the subject 102 may view the vision test output by the external medium 110. The subject 102 may enter an input signal into the feedback device 116. Based on the input signal, the feedback device 116 may generate feedback 204 that indicates the subject's perception, reaction, performance, or a combination thereof, of the vision test. The feedback device 116 may provide the feedback 204 to the vision screening device 104, such as via a Bluetooth connection and/or NFC, as described above. In other examples, the feedback device 116 may display the feedback 204 via the feedback device 116, such as a user interface (UI). In some examples, the display associated with the feedback device 116 may include one or more selectable options which may allow the user 106 and/or the subject 102 to send the results 206, such as to the EMR system 118 or the web server 120.

According to examples, the vision screening device 104 may generate a result 206 based on the key 202 and the feedback 204. For instance, the key 202 may correspond to the vision test being administered to the subject 102. In some examples, the key 202 may be one or multiple keys which may be uploaded to the test datastore 144 such that the vision screening device 104 may determine the result 206 of the test. For instance, based on receiving the feedback 204 from the test, the vision screening device 104 may compare the key 202 and the feedback 204. In some cases, the result 206 indicates a discrepancy between the key 202 and the feedback 204. For instance, if the key 202 is a shape that is within a threshold distance of a symbol, and the feedback 204 is an attempt by the subject to trace the symbol, then the discrepancy may be a number of times and/or an amount that the feedback 204 moves outside of the key 202. In various implementations, the vision screening device 104 determines whether the subject is suspected to have an ocular condition based on the discrepancy between the key 202 and the feedback 204. The result 206 may indicate whether the subject is suspected to have the ocular condition. The vision screening device 104 may store the result 206, output the result 206 to a user, transmit a signal including the result 206 to an external device, or a combination thereof. In some examples, the key 202 may be updated and/or removed based on the test being administered and/or the subject taking the test.

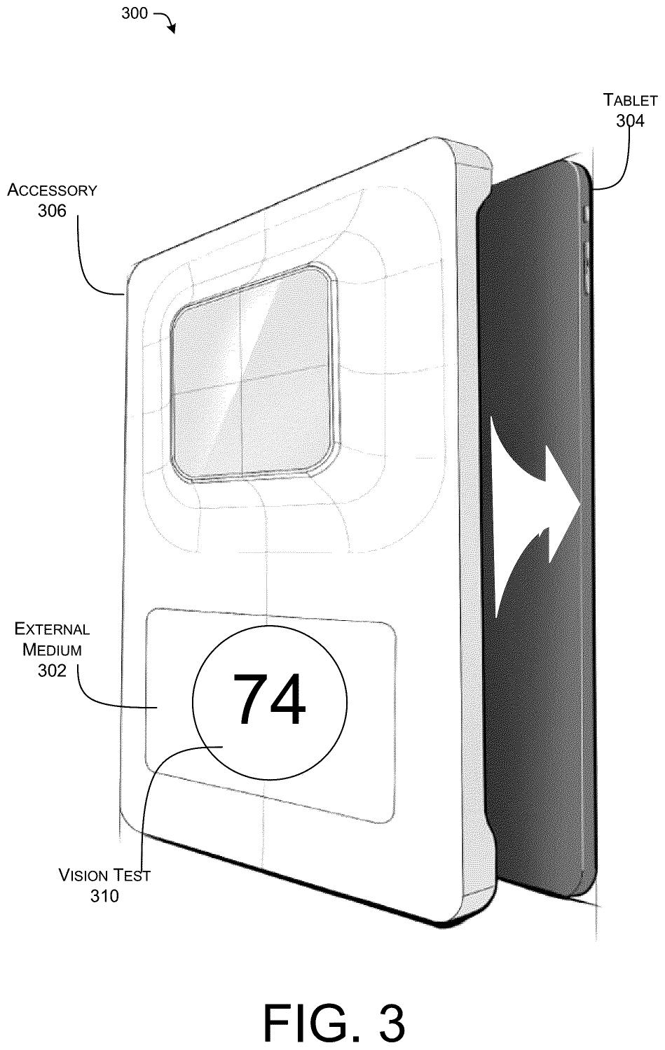

FIG. 3 illustrates an example vision screening device 300 with an external medium 302 for administering a vision test to a subject. In particular cases, the vision screening device 300 includes a tablet computer 304 that is mechanically coupled to an accessory 306. The accessory 306 includes the external medium 302 that displays a vision test 310. In various implementations, a screen of the tablet computer 304 faces the user of the vision screening device 300 and the vision test 310 faces the subject of the vision screening device 300. In some cases, the accessory 306 indicates the code of the vision test 310 by transmitting an NFC and/or RFID signal to the tablet computer 304.

For example, the vision screening device 300 illustrated in FIG. 3 may be verbal test in which the subject of the vision screening 300 is presented, via the display of the external medium 302, a symbol, such as a number. For example, the current illustration depicts the number 74 being displayed via the external medium. The subject may then be instructed, such as by a user of the vision screening device, to verbalize the number being projected. The user administering the vision test may then indicate, via the tablet 304, whether the subject verbalized the correct number.

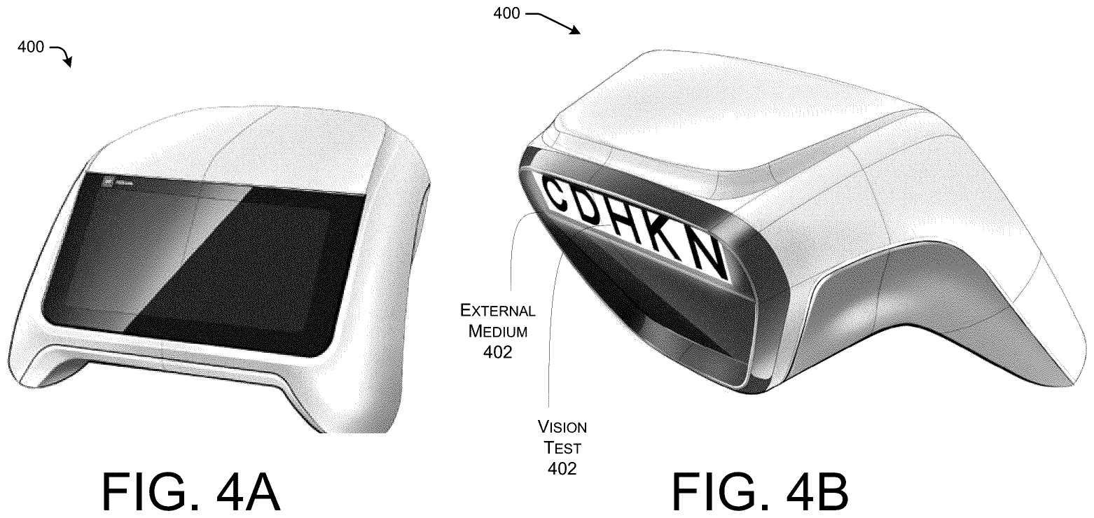

FIGS. 4A and 4B illustrate another example vision screening device 400 with an external medium 402 for administering a vision test to a subject. FIG. 4A illustrates a side of the vision screening device 400 that faces a subject. FIG. 4B illustrates a side of the vision screening device 400 that faces a user. The vision screening device 400 may be a standalone device that is configured to be held by the user. The user may operate the vision screening device 400 via a user interface (e.g., a touchscreen). In various implementations, the external medium 402 of the vision screening device 400 includes a substrate that displays a vision test 404 to the subject. For example, similar to the vision test described above in FIG. 3, the subject may be asked, by an administrator of the vision test, to verbalize the symbol that are being presented to the subject via the vision screen device 400.

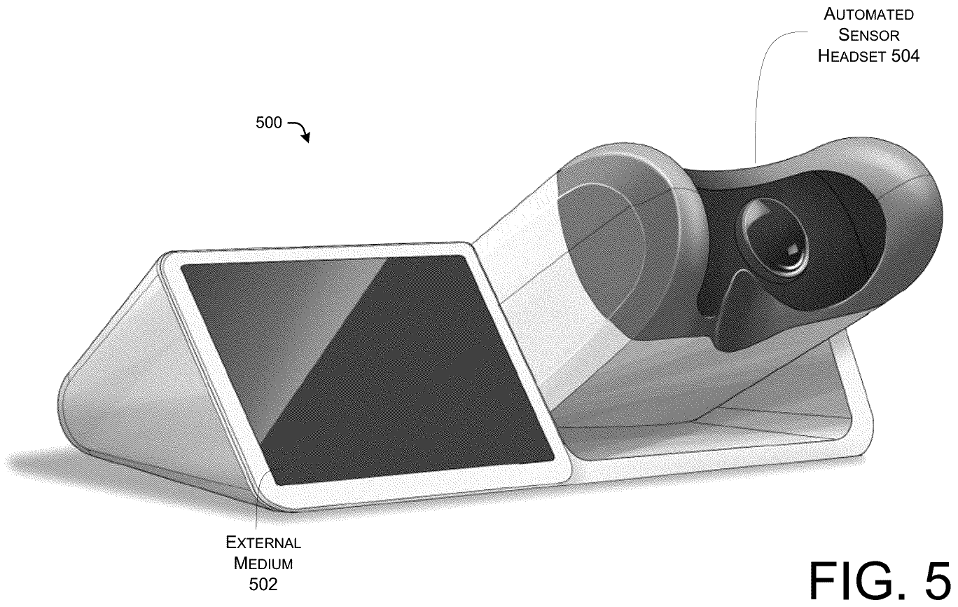

FIG. 5 illustrates a further example vision screening device 500 with an external medium 502 for administering a vision test to a subject. In this example, the vision screening device 500 can be operated while being disposed on a tabletop or other horizontal surface. The external medium 502, for example, may be a screen that displays a vision test to a subject. Further, the vision screening device 500 includes an automated sensor headset 504. When the subject brings their eyes to the automated sensor headset 504, the vision screening device 500 may perform an automated vision test (e.g., autorefraction test, red reflex test, etc.) on the subject. Based at least in part on a determination that the automated vision test is complete, the vision screening device may cause presentation, via the external medium of the vision screening device 500, of one or more results associated with the vision test. In some examples, the external medium may contain a user interface element which may be configured to receive instructions, such as by the subject and/or an administrator of the test, to send the results of the vision screen to a different location, such as the web server 120.

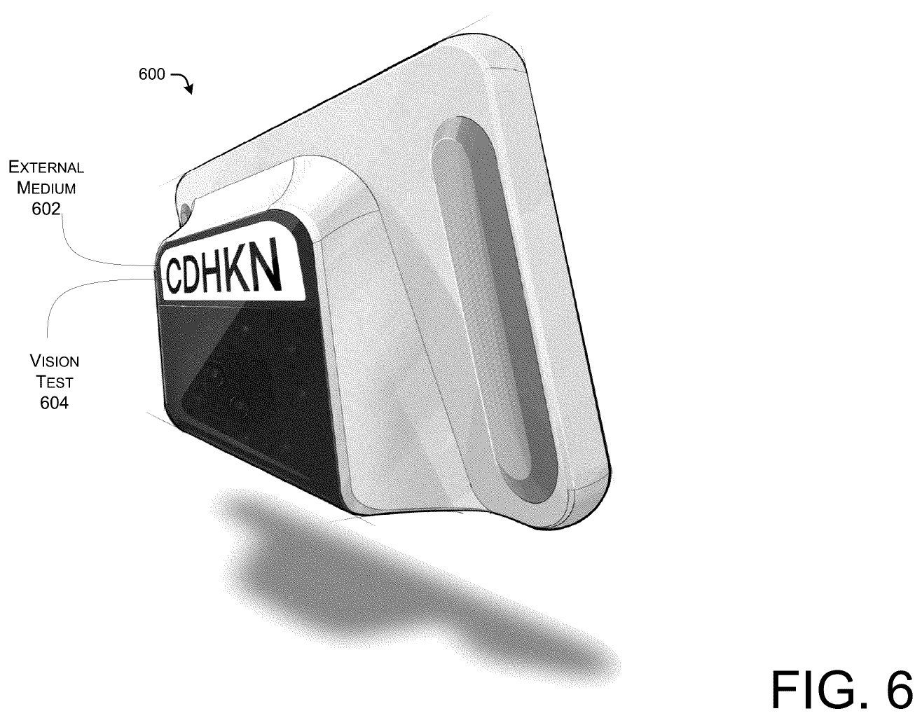

FIG. 6 illustrates an additional example vision screening device 600 with an external medium 602 for administering a vision test to a subject. In this example, the vision screening device 600 includes a handheld tablet computer. The vision screening device 600 may also have a flat surface that allows the vision screening device 600 to rest on a tabletop or horizontal surface. The external medium 602 may face a subject, so that the subject may view a vision test 604 displayed by the external medium 602. Although not specifically illustrated, the vision screening device 600 may further include a touchscreen or other user interface that can be operated by a user, similar to that described above with respect to vision screening devices 300, 400, and 500.

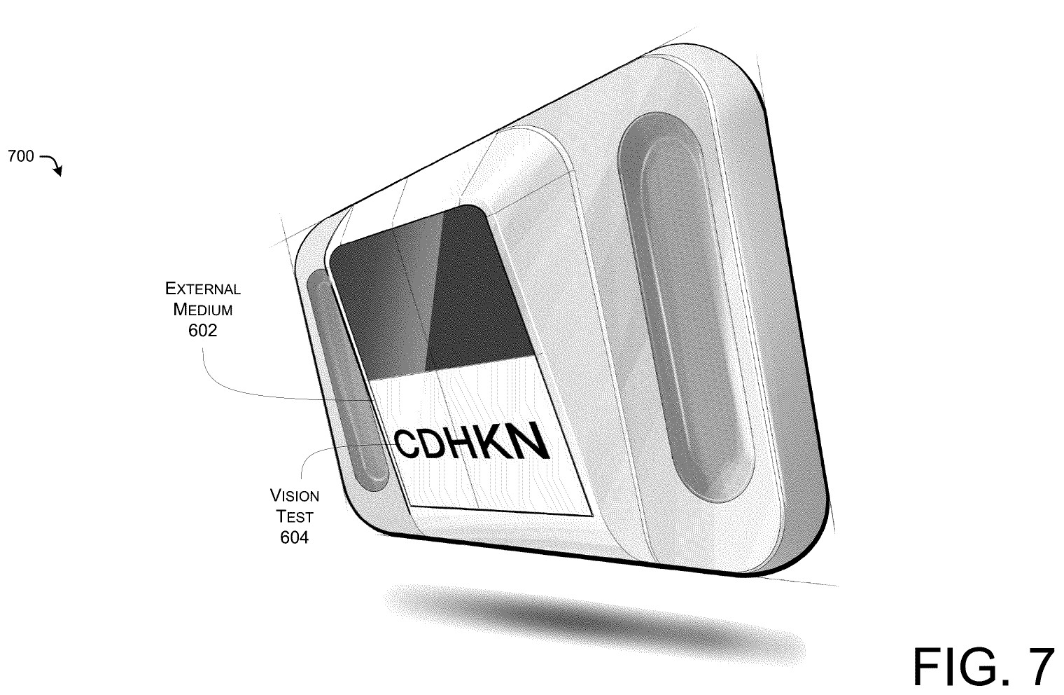

FIG. 7 illustrates yet another example vision screening device 700 with an external medium 702 for administering a vision test to a subject. In this example, the vision screening device 700 includes a handheld tablet computer. The external medium 702 may face a subject, so that the subject may view a vision test 704 displayed by the external medium 702. Although not specifically illustrated, the vision screening device 700 may further include a touchscreen or other user interface that can be operated by a user.

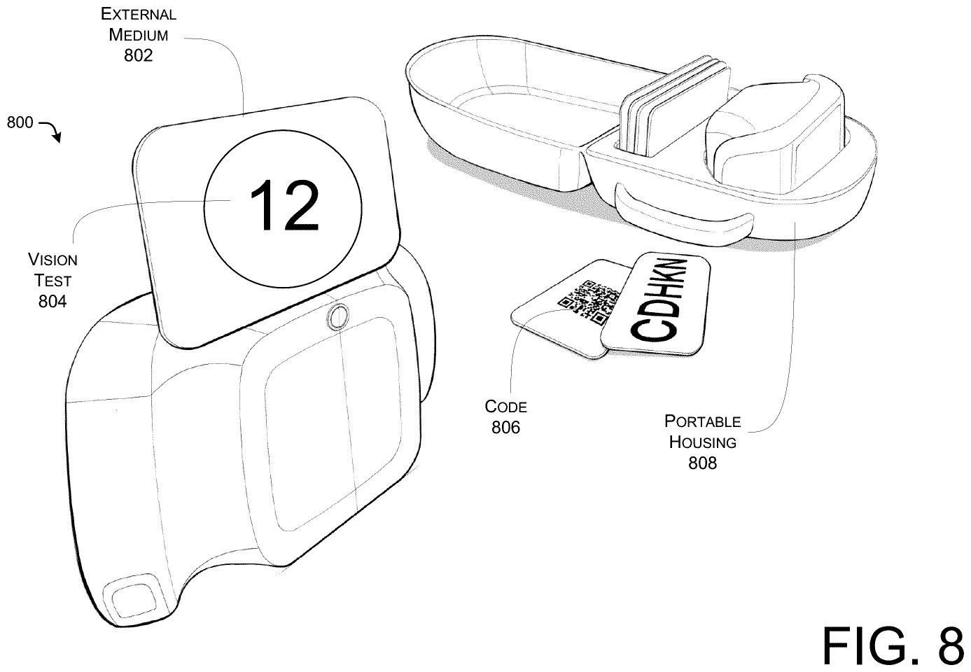

FIG. 8 illustrates an example vision screening device 800 in which a removeable external medium 802 is mounted on the vision screening device 800. The external medium 802, for example, is one of multiple cards that can be selectively attached to the vision screening device 800. The external medium 802 displays a vision test 804 to a subject. In various cases, a different code 806 is printed on each of the cards (including the external medium 802), which can be detected by the vision screening device 800 (e.g., using a camera) and used to identify the vision test 804. For example, the current embodiment illustrates the code 806 as a QR code. For example, prior to administering the test, an administrator of the test may, using a camera of the vision screening device 800 (not illustrated) scan the code 806. Based at least in part on receiving the code 806, the vision screening device 800 may determine a vision test associated with the code 806. Accordingly, the multiple cards can respectively display different vision tests that can be used by the vision screening device 800. Additionally, or alternatively, the code may be used to determine a key associated with the test such that the vision screening device 800 may ensure that the results of the vision test 804 correspond to the correct code as the key being used to determine the results of the vision test 804. In some cases, the vision screening device 800, as well as the cards, can be packaged into a portable housing 808 that has a handle for ease of transport.

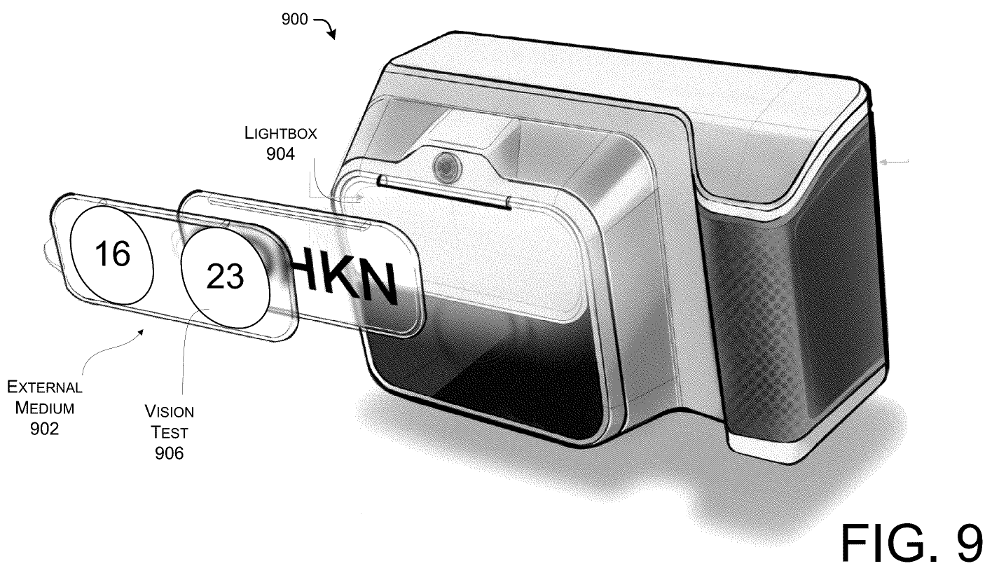

FIG. 9 illustrates another example vision screening device 900 with removeable external medium 902 that can be selectively attached to the vision screening device 900. In this example, the external media 902 include transparent slides that can be mounted on a lightbox 904 of the vision screening device 900. A subject can view a vision test 906 on an example external medium 902 when light emitted from the lightbox 904 is transmitted through the external medium 902. In some cases, vision screening device 900 detects a code from an example external medium 902 via an RFID and/or NFC signal transmitted between the external medium 902 and the vision screening device 900.

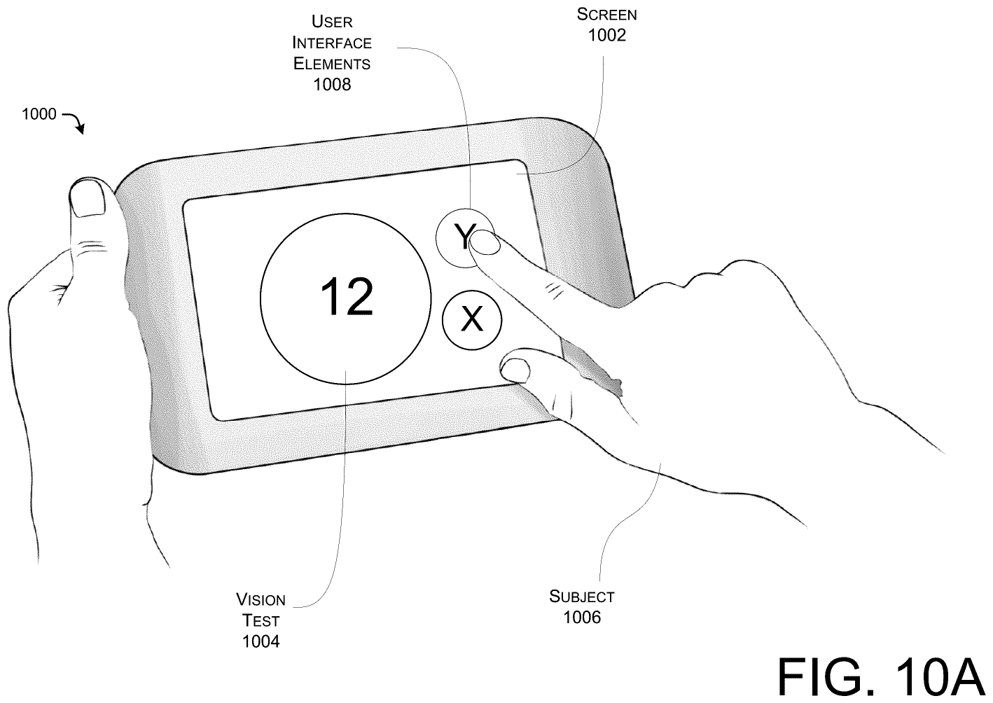

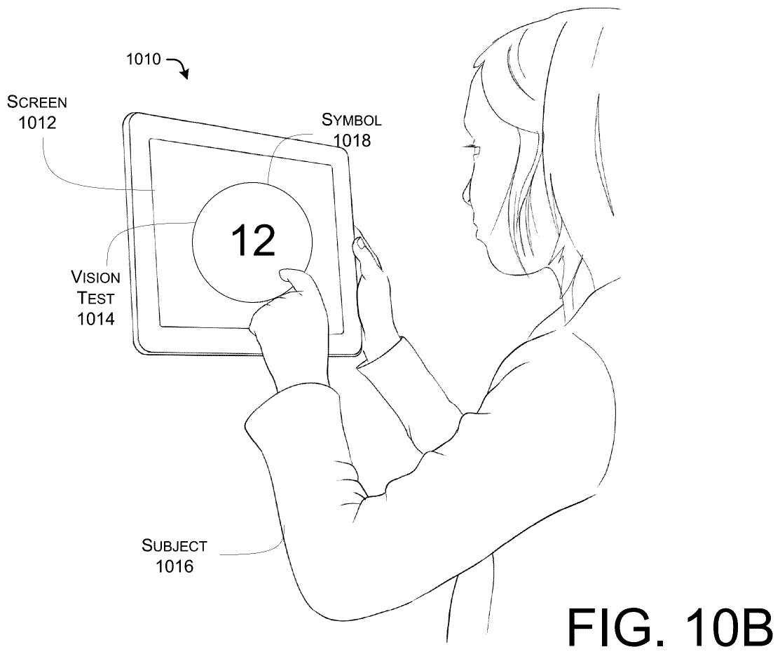

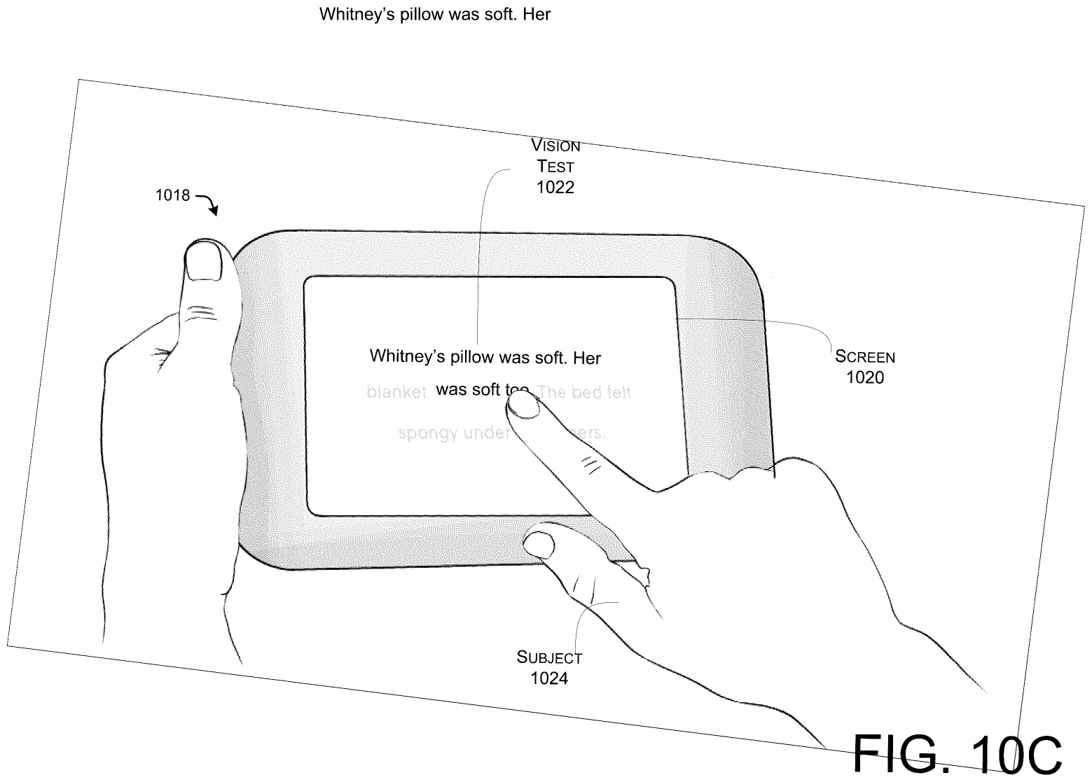

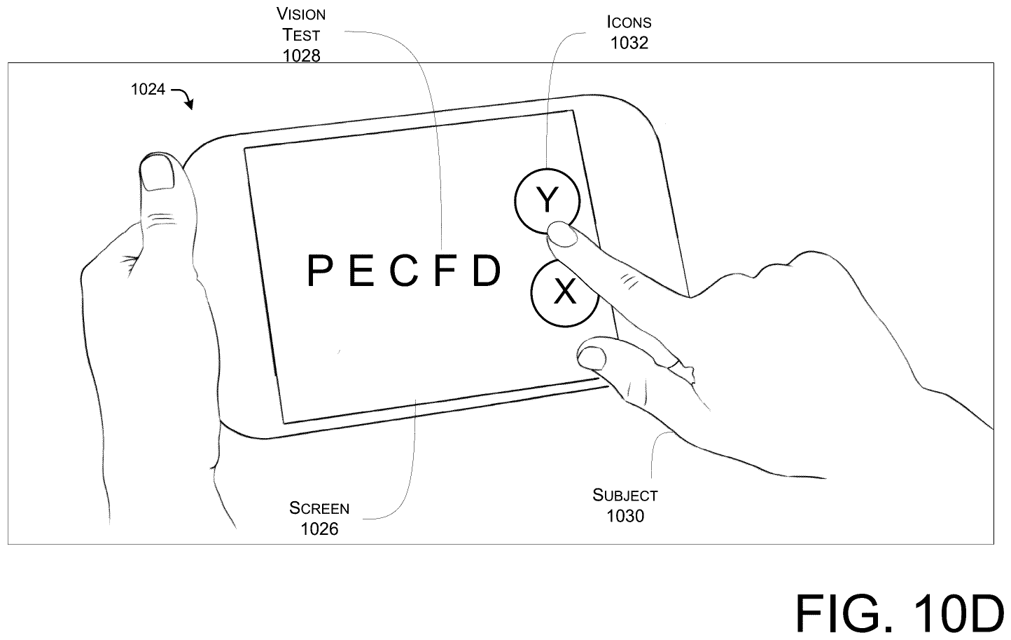

FIGS. 10A to 10D illustrate examples of feedback devices including external media for administering a vision test to a subject. FIG. 10A illustrates an example feedback device 1000 including a screen 1002 that outputs a vision test 1004 to a subject 1006. As described above, a vision screening test may require the subject 1006 to identify whether one or more elements in a user interface of the feedback device correspond with an element provided by a user, or administrator of the vision test. For example, the vision test illustrated in FIG. 10A illustrates user interface elements 1008 displayed on the screen 1002, such as the number “12,” as well as a “Y” (corresponding to “Yes”) and a “N” (corresponding to “No”). Based at least in part on the user audibly saying a number out loud to the subject 1006 may select the “Y” or the “N” in a determination that the number said by the user corresponds with the number on the screen 1002. In the current illustration, the subject 1006 has selected the “Yes” option.

FIG. 10B illustrates an example feedback device 1010 including a screen 1012 that outputs a vision test 1014 to a subject 1016. As described above, a vision screening test may require the subject 1016 to trace a symbol 1018 as it appears on the feedback device 1010. For example, the vision test illustrated in FIG. 10B illustrates a symbol 1018 displayed on the screen 1012, such as the number “12”. Based at least in part on the user prompting different symbols 1018 to appear on the screen 1012, the subject 1016 may input feedback into the feedback device 1010 by tracing the symbol 1018 of the vision test 1014. In the current illustration, the subject 1016 has begun tracing the symbol 1018, number “12”. The tracing by the subject 1016 can be detected via one or more touch sensors integrated with the screen 1012. In other examples, the touch sensors may detect a selection of one or more items on the screen 1012.

FIG. 10C illustrates an example feedback device 1018 including a screen 1020 that outputs a vision test 1022 (e.g., a reading speed test) to a subject 1024. As described above, a vision test 1022, such as a reading speed test, may require the subject 1024 to identify one or more words, phrases, or sentences. For example, the vision test illustrated in FIG. 10C illustrates sentences 1022 displayed on the screen 1020, such as “Whitney's pillow was soft,” as well as “Her blanket was soft too.” Based at least in part on the user prompting different words, phrases, or sentences of the vision test 1022 to appear on the screen 1020, the subject 1024 inputs feedback into the feedback device 1018 by touching words 1020 within the vision test 1022, which can be detected via one or more touch sensors integrated with the screen 1020. In the current illustration, the subject 1024 has tapped the word “too”.

FIG. 10D illustrates an example feedback device 1024 including a screen 1026 that outputs a vision test 1028 (e.g., a close vision test) to a subject 1030. In some examples, a vision screening test may require the subject 1030 to identify whether one or more elements in the vision test 1028 correspond with an element provided by a user or administrator of the vision test. For example, the vision test illustrated in FIG. 10D illustrates a vision test 1028, such as “PECFD,” as well as icons 1032, such as a “Y” (corresponding to “Yes”) and a “X” (corresponding to “No”), displayed on the screen 1026. Based at least in part on the user audibly saying a letter out loud, the subject 1030 may select the “Y” or the “X” in a determination that the letter(s) said by the user corresponds with the letter(s) on the screen 1026. In the current illustration, the subject 1030 has selected the “Yes” option. The touch of the subject 1030, in various cases, may be detected via one or more touch sensors integrated with the screen 1026.

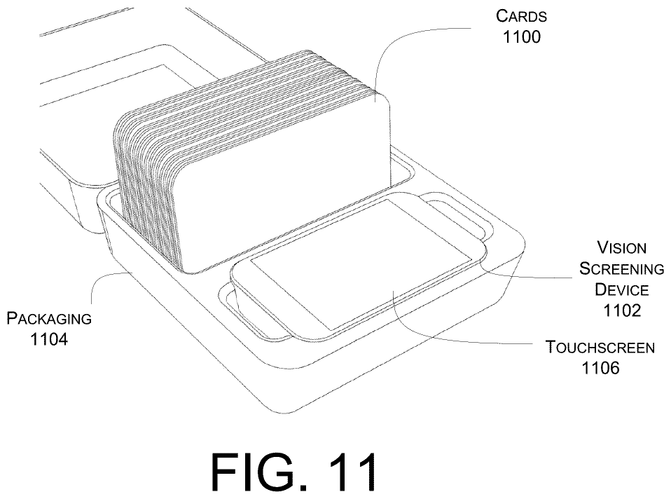

FIG. 11 illustrates a vision screening device 1102 packaged with cards 1100 that serve as external media. Packaging 1104 is configured to hold the vision screening device 1102 and the cards 1100. In various implementations, each card 1100 is a printed substrate that displays a particular vision test. In some cases, the cards 1100 respectively display different vision tests. The vision screening device 1102 includes a touchscreen 1106. An example card is selected and attached to a surface of the vision screening device 1102 that is opposite of the touchscreen 1106. In some implementations, the vision screening device 1102 detects the code of the selected card by receiving an NFC and/or RFID signal from the card. In some cases, the vision screening device 1102 includes a camera that captures an image of the code printed on the card before the card is attached to the vision screening device 1102 or while the card is attached to the vision screening device 1102.

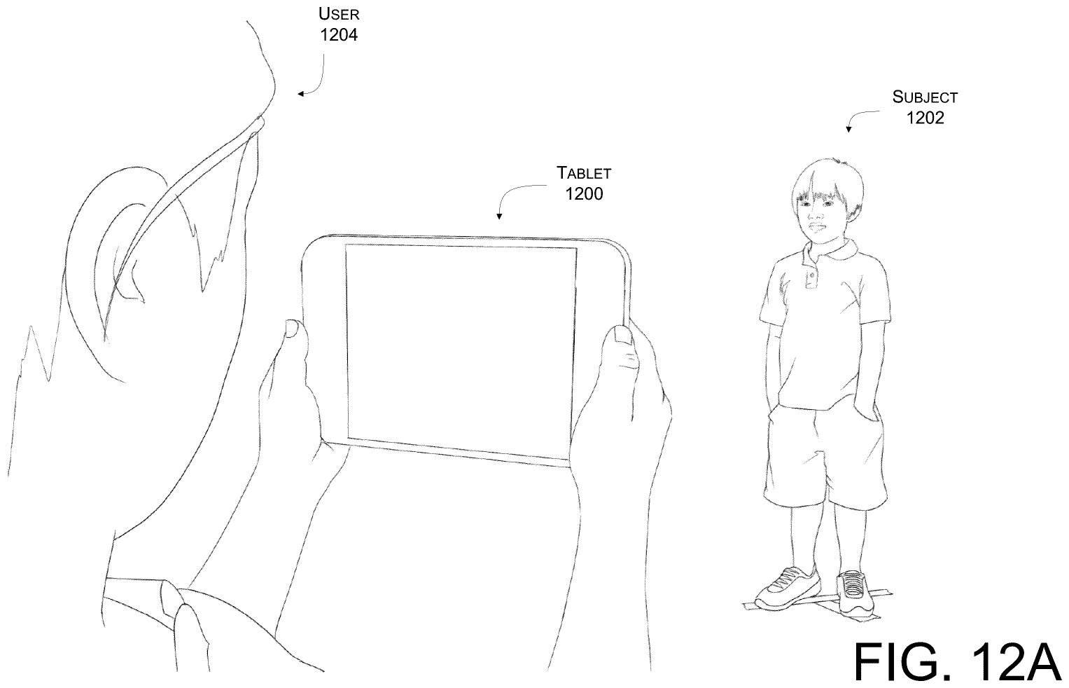

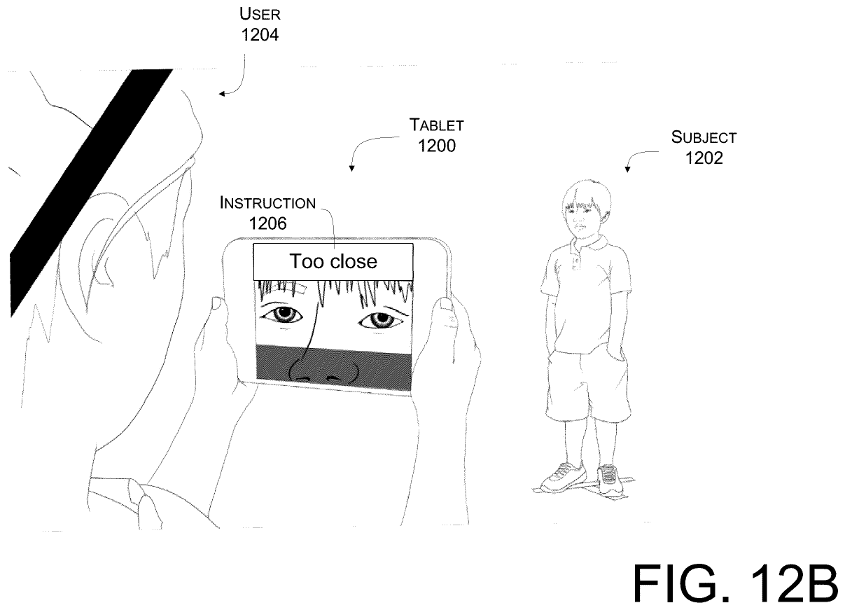

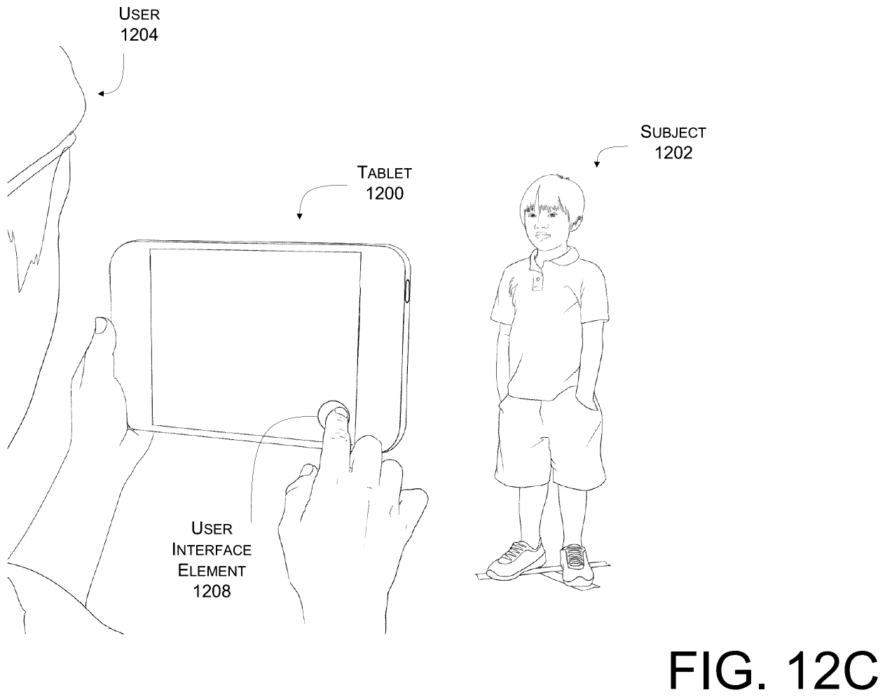

FIGS. 12A to 12C illustrate an example workflow for administering a vision test to a subject using a tablet 1200 as an external medium and feedback device. FIG. 12A illustrates the tablet 1200 being used to assess a subject 1202. As described above, a user 1204 may hold the tablet 1200 at a distance away from the subject 1202, such that the subject 1202 may view a vision test displayed by the table 1200. In some examples, the distance between the tablet 1200 and the subject 1202 may be necessary based on the vision test being administered. For example, the vision test being administered may require the tablet 1200 to be placed at pre-determined distance from the subject 1202 in order to obtain accurate results. In some example, the tablet 1200 may include one or more cameras which may be capable of determining a distance from the cameras to the subject 1202. For example, the camera may take an image of the subject 1204. Based at least in part on the image, a processor of the tablet 1200 may determine a distance from the tablet 1200 to the subject 1202. Based in least in part on receiving an indication of selection of a vision test to be used to assess the subject 1202, the tablet 1200 may determine a preferred distance that the subject 1202 must be from the tablet 1200. The tablet 1200 may compare the distance from the subject 1202 to the tablet to the preferred distance to determine whether the subject 1202 is too far from or too close to the tablet 1200.

In some examples, the user 1204 may physically select and/or placing a vision test printed on a card onto to the tablet 1200 side facing the subject 1202. Additionally or alternatively, the user 1204 may select a vision test from the various vision tests capable of being displayed on the tablet 1200. Based at least in part on the user 1204 presenting the vision test to the subject 1202, the subject 1202 can audibly respond to prompts from the user 1204 or can be observed for bodily behaviors, among other response actions. In the current illustration, the subject 1202 is standing at a distance away from the user 1204 and the testing tablet 1200 where the user 1204 has yet to select a vision test to be administered.

FIG. 12B illustrates the tablet 1200 providing an instruction 1206 to the user 1204 to facilitate the vision test. In various implementations, the tablet 1200 may include a camera that captures at least one image of the subject 1202. Based on the image(s), the tablet 1200 may determine whether the subject 1202 is too close or too far from the tablet 1200 for the vision test, as described above in FIG. 12A. Based at least in part on the user 1204 selecting a specific vision test, the tablet 1200 outputs the instruction 1206 to establish an appropriate distance between the tablet 1200 and the subject 1202 to perform the vision test properly. In the current illustration, the user 1204 has been given an instruction 1206 via the tablet 1200 that the subject 1202 is “Too close,” for the selected vision test to be administered properly.

FIG. 12C illustrates the user 1204 inputting feedback into the tablet 1200 via a user interface element 1208. For example, the subject 1202 may answer auditory prompts from the user 1204 regarding the administered vision test, such as how the subject 1202 perceives the test. In some examples, the answers from the subject 1202 may be manually input into the tablet 1200 via the user interface element 1208 by the user 1204. For example, the subject 1202 may speak about how they perceive the vision test. In response, the user 1204 may touch a user interface element 1208 on the screen of the tablet 1200. The tablet 1200 may detect the feedback by detecting the touch of the user 1204 on the screen. In other examples, the tablet 1200 may automatically receive the answers from the subject 1202. For example, the tablet 1200 may include one or more speakers which may be configured to receive audio and translate that audio to text. The tablet 1200 may then store the input as feedback. which are then cataloged via the user 1204 selecting the appropriate user interface element(s) 1208.

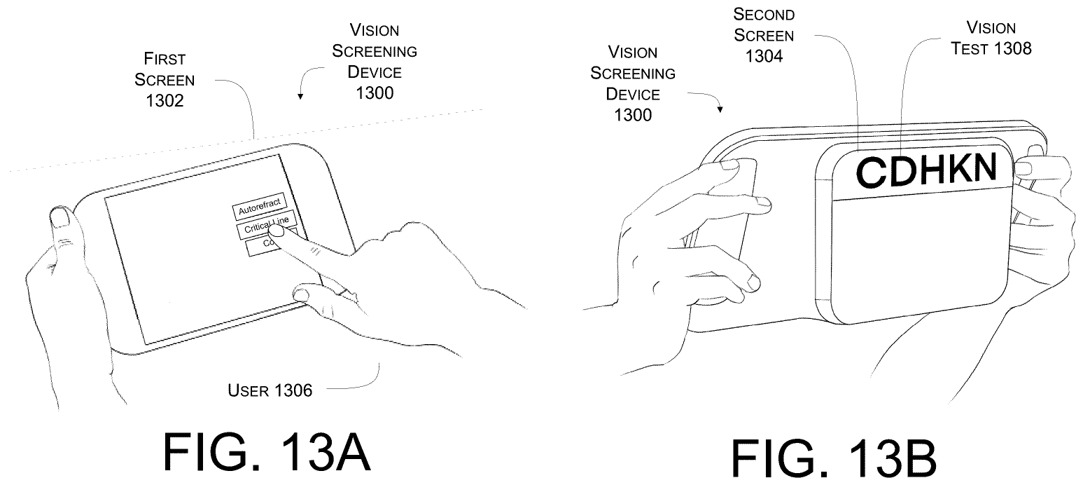

FIGS. 13A and 13B illustrate an example workflow for vision screening using a vision screening device 1300 that includes a first screen 1302 and a second screen 1304. FIG. 13A illustrates the first screen 1302 of the vision screening device, which may be a touchscreen that is displayed to a user 1306. The user 1306 may operate the vision screening device 1300 by touching the first screen 1302. For example, the user 1306 may select a vision test 1308 for screening a subject by touching an icon displayed on the first screen 1302.

FIG. 13B illustrates the vision test 1308 output by the second screen 1304. As shown, the second screen 1304 may be on a different surface of the vision screening device 1300 than the first screen 1302. Accordingly, the subject may view the vision test 1308 while the user 1306 is viewing the first screen 1302.

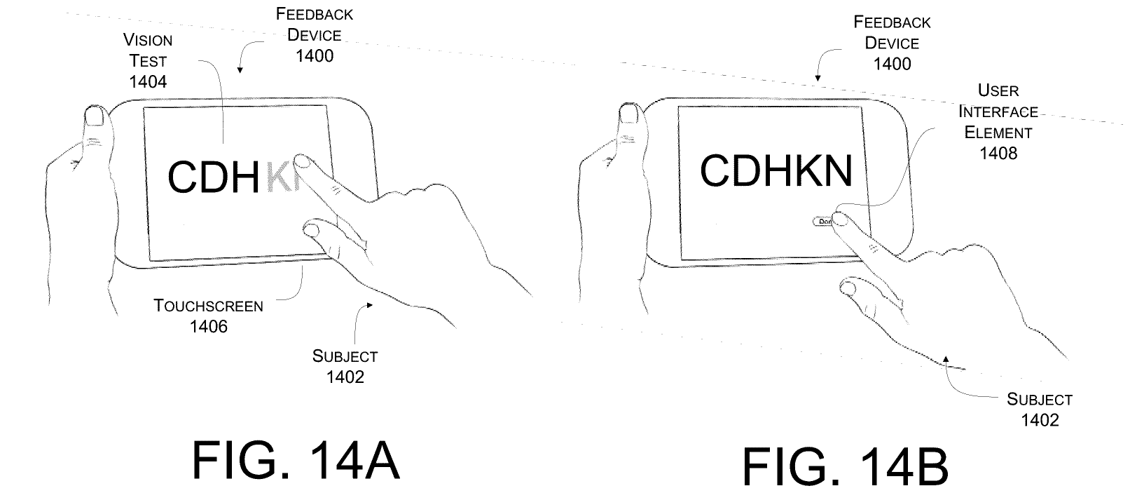

FIGS. 14A and 14B illustrate a feedback device 1400 configured to receive feedback directly from a subject 1402. FIG. 14A illustrates the feedback device 1400 outputting vision test 1404 to the subject 1402 on a touchscreen 1406. The vision test 1404 includes multiple symbols. The subject 1402 inputs feedback about the vision test 1404 into the feedback device 1400 by tracing the symbols displayed on the touchscreen 1406. For example, one or more touch sensors integrated with the touchscreen 1406 detect the touch of the subject 1402. FIG. 14B illustrates another screen output on the touchscreen 1406 that displays a user interface element 1408 (e.g., an icon). The subject 1402 may input feedback by touching the user interface element 1408. For example, the feedback device 1400 determines that the subject 1402 has finished the vision test 1404 by detecting that the subject 1402 has touched the user interface element 1408.

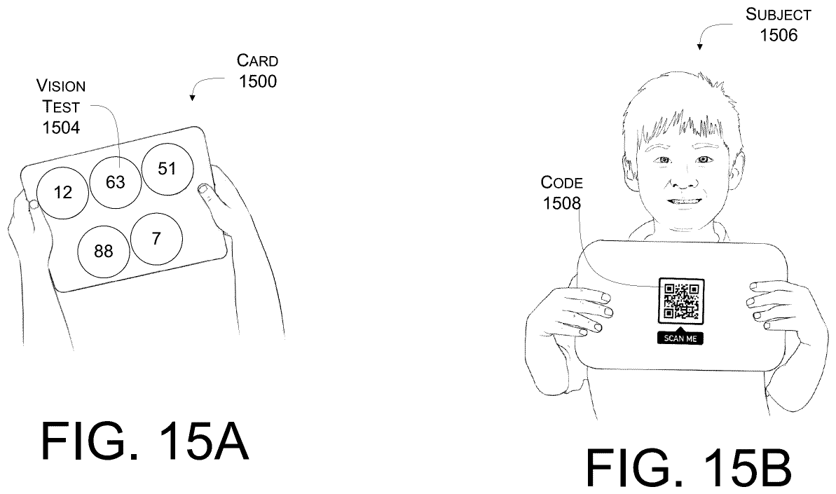

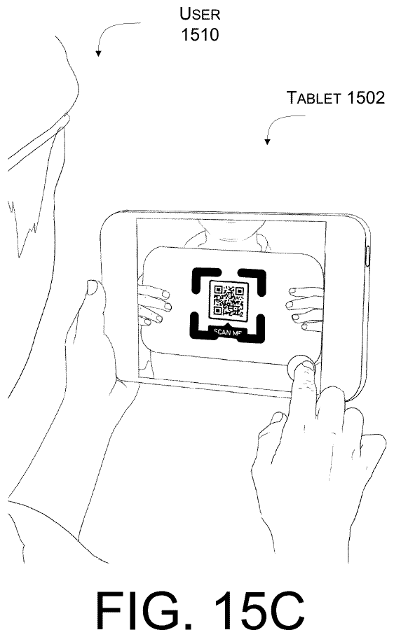

FIGS. 15A to 15C illustrate a workflow for vision screening in which a handheld card 1500 is used as an external medium and a tablet 1502 is used as a feedback device. FIG. 15A illustrates a vision test 1504 printed on a side of the card 1500. Although color is not illustrated in the current illustration, in particular implementations, the vision test 1504 includes a color vision test. FIG. 15B illustrates a subject 1506 holding the card 1500. In particular, the side of the card 1500 that displays the vision test 1504 may be facing the subject 1506, such that the subject 1506 can view the vision test 1504. A code 1508 may be displayed on another side of the card 1500, such that the code 1508 may be facing outward as the subject 1506 is viewing the vision test 1504. In some examples, the code 1506 may be facing a user or administrator of the test, such that the user or administrator of the test may scan the code 1506 with a device, such as a tablet, as described below. For example, FIG. 15C illustrates a user 1510 operating the tablet 1502. In various implementations, the tablet 1502 may include a camera that is configured to capture at least one image of the code 1508 displayed on the card 1500. Based on the code 1508, the tablet 1502 may identify the vision test 1504 that is displayed on the card 1500. Thus, upon receiving feedback from the subject 506 regarding the test, the tablet 1502 and/or the user 1510 may enter the feedback into the tablet 1502.

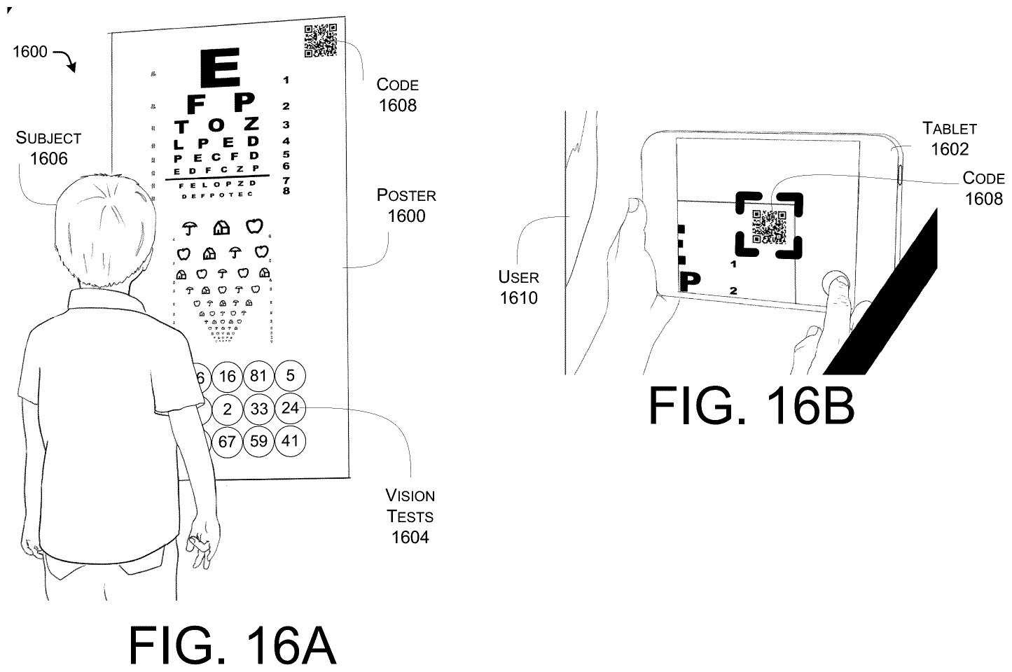

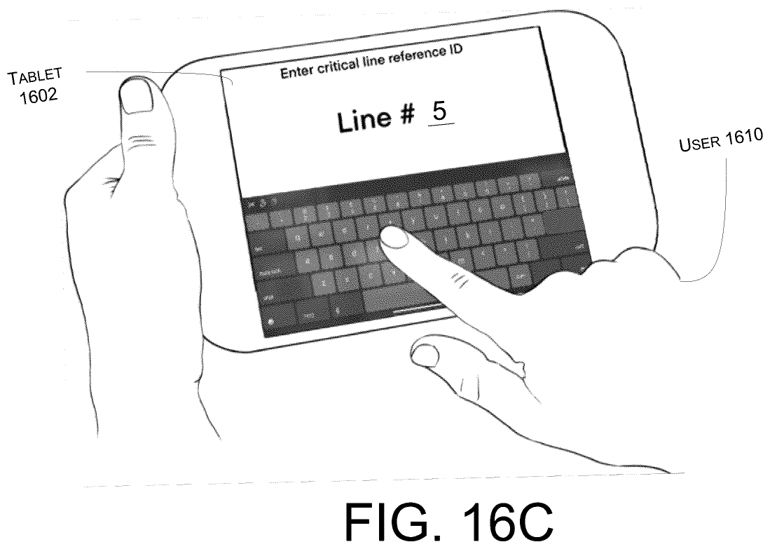

FIGS. 16A to 16C illustrate a workflow for vision screening in which a poster 1600 is used as an external medium and a tablet 1602 is used as a feedback device. FIG. 16A illustrates a vision test 1604 printed on the poster 1600, which can be viewed by a subject 1606. In particular, multiple vision tests 1604 may be printed on the poster 1600. However, a poster is merely an example embodiment and vision tests may be displayed on any external medium, such as projected via a screen, on a card, or on paper, to name a new non-limiting examples. In addition, the poster 1600 may display a code 1608 which may allow results associated with the test to be accurately scored and associated with the subject 1606. FIG. 16B illustrates a user 1610 operating the tablet 1602. The tablet 1602 may have a camera configured to capture at least one image of code 1608 displayed on the poster 1600. Based on the code 1608, the tablet 1602 may identify the vision test(s) 1604 being viewed by the subject 1606. FIG. 16C illustrates an example of the user 1610 inputting feedback about the perception of the vision test 1604 by the subject 1606. For example, the subject 1606 may at least attempt to audibly read a line of symbols in the vision test(s) 1604, the user 1610 may determine that the subject 1606 incorrectly read at least one of the symbols, and the user 1610 may indicate the incorrectly read line into the tablet 1602 as feedback. The tablet 1602, in some cases, may store or output the feedback. In some cases, the tablet 1602 may determine a condition of the subject 1606 based on the feedback and may store and/or output an indication of the condition.

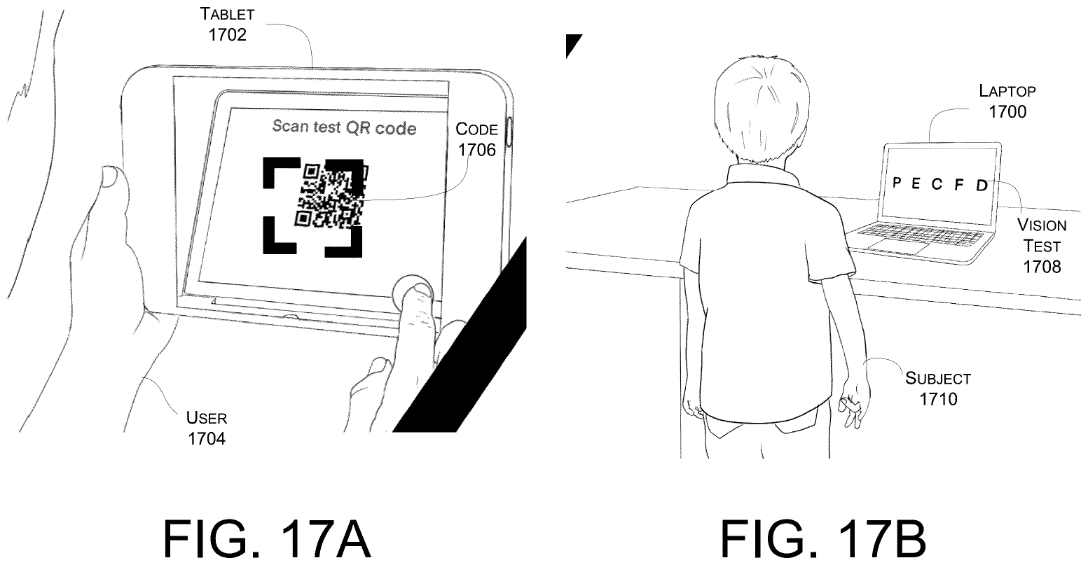

FIGS. 17A and 17B illustrate a workflow for vision screening in which a laptop 1700 is used as an external medium and a tablet 1702 is used as a feedback device. FIG. 17A illustrates a user 1704 operating the tablet 1702 at a first time. The tablet 1702 may have at least one camera configured to capture an image of a screen of the laptop 1700 as the screen is displaying a code 1706. FIG. 17B illustrates a vision test 1708 output by the laptop 100 at a second time. In various cases, the tablet 1702 may identify the vision test 1708 based on the code 1706 displayed on the laptop 1700. In various implementations, a subject 1710 may view the vision test 1708 and provide feedback on the vision test 1708. In some cases, the user 1704 and/or the subject 1710 inputs the feedback into the tablet 1702.

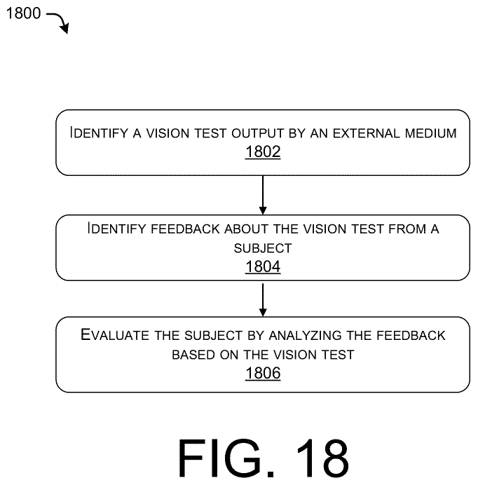

FIG. 18 illustrates an example process 1800 for vision screening using external media. The process 1800 may be performed by an entity, such as at least one processor, the vision screening device 104, the external medium 110, the test datastore 114, the feedback device 116, or any combination thereof.

At 1802, the entity identifies a vision test output by an external medium. For example, the entity may receive a signal from the external medium. The signal may be a wireless signal (e.g., an RFID signal, an NFC signal, etc.) or light, in some cases. In various implementations, the signal is indicative of a code associated with the vision test. For instance, the entity captures an image of a QR code or other type of barcode that is uniquely associated with the vision test and displayed by the external medium. In various implementations, the external medium is a passive medium, such as a card, a poster, or other type of printed substrate. In some cases, the external medium is a device, such as a mobile phone, a tablet computer, a VR headset, or a laptop computer. The vision test, for instance, includes at least one of a color vision test, a reading comprehension test, a concussion test, a near vision test, a reading speed test, or a visual acuity test.

At 1804, the entity identifies feedback about the vision test from a subject. In some implementations, the feedback is directly received by the entity from the subject. For example, the entity identifies the feedback by detecting the subject tracing a shape on the surface of a screen (e.g., detected using one or more touch sensors), the subject touching an icon displayed on the screen, or a voice of the subject indicating the feedback. In some implementations, the entity receives the feedback from a user who is not the subject or by receiving a signal from an external device that detected the feedback.

At 1806, the entity evaluates the subject by analyzing the feedback based on the vision test. In various implementations, the entity may determine whether the subject is suspected to have at least one ocular condition by analyzing the feedback in view of the vision test. In some cases, the entity identifies a key associated with the vision test and compares the key to the feedback. The entity may compare a discrepancy between the key and the feedback to one or more thresholds. For example, if the discrepancy is above a first threshold or below a threshold, the entity may determine whether the subject is suspected to have at least one ocular condition. In some implementations, the entity may store, transmit, and/or output an indication of whether the subject is suspected to have the ocular condition.

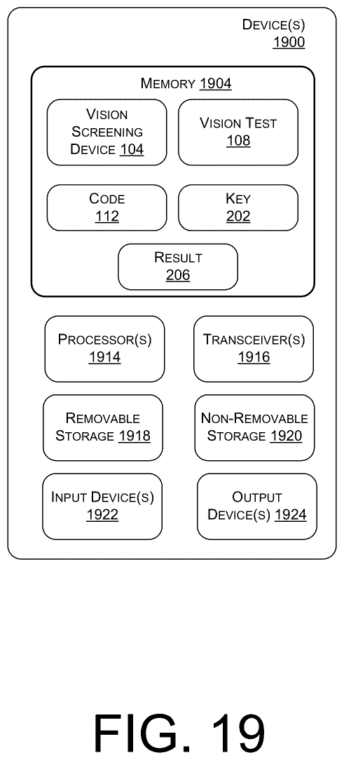

FIG. 19 illustrates at least one example device 1900 configured to enable and/or perform the some or all of the functionality discussed herein. Further, the device(s) 1900 can be implemented as one or more server computers 1902, a network element on a dedicated hardware, as a software instance running on a dedicated hardware, or as a virtualized function instantiated on an appropriate platform, such as a cloud infrastructure, and the like. It is to be understood in the context of this disclosure that the device(s) 1900 can be implemented as a single device or as a plurality of devices with components and data distributed among them.

As illustrated, the device(s) 1900 comprise a memory 1904. In various embodiments, the memory 1904 is volatile (including a component such as Random Access Memory (RAM)), non-volatile (including a component such as Read Only Memory (ROM), flash memory, etc.) or some combination of the two.

The memory 1904 may include various components, such as at least of the vision screening device 104, the vision test 108, the code 112, the key 202, or the result 206. Any of the vision screening device 104, the vision test 108, the code 112, the key 202, or the result 206 can include methods, threads, processes, applications, or any other sort of executable instructions. The vision screening device 104, the vision test 108, the code 112, the key 202, or the result 206 and various other elements stored in the memory 1904 can also include files and databases.

The memory 1904 may include various instructions (e.g., instructions in the vision screening device 104, the vision test 108, the code 112, the key 202, or the result 206), which can be executed by at least one processor 1914 to perform operations. In some embodiments, the processor(s) 1914 includes a Central Processing Unit (CPU), a Graphics Processing Unit (GPU), or both CPU and GPU, or other processing unit or component known in the art.

The device(s) 1900 can also include additional data storage devices (removable and/or non-removable) such as, for example, magnetic disks, optical disks, or tape. Such additional storage is illustrated in FIG. 19 by removable storage 1918 and non-removable storage 1920. Tangible computer-readable media can include volatile and nonvolatile, removable and non-removable media implemented in any method or technology for storage of information, such as computer readable instructions, data structures, program modules, or other data. The memory 1904, removable storage 1918, and non-removable storage 1920 are all examples of computer-readable storage media. Computer-readable storage media include, but are not limited to, RAM, ROM, EEPROM, flash memory or other memory technology, CD-ROM, Digital Versatile Discs (DVDs), Content-Addressable Memory (CAM), or other optical storage, magnetic cassettes, magnetic tape, magnetic disk storage or other magnetic storage devices, or any other medium which can be used to store the desired information and which can be accessed by the device(s) 1900. Any such tangible computer-readable media can be part of the device(s) 1900.

The device(s) 1900 also can include input device(s) 1922, such as a keypad, a cursor control, a touch-sensitive display, voice input device, etc., and output device(s) 1924 such as a display, speakers, printers, etc. These devices are well known in the art and need not be discussed at length here. In particular implementations, a user can provide input to the device(s) 500 via a user interface associated with the input device(s) 1922 and/or the output device(s) 1924.

As illustrated in FIG. 19, the device(s) 1900 can also include one or more wired or wireless transceiver(s) 1916. For example, the transceiver(s) 1916 can include a Network Interface Card (NIC), a network adapter, a LAN adapter, or a physical, virtual, or logical address to connect to the various base stations or networks contemplated herein, for example, or the various user devices and servers. To increase throughput when exchanging wireless data, the transceiver(s) 1916 can utilize Multiple-Input/Multiple-Output (MIMO) technology. The transceiver(s) 1916 can include any sort of wireless transceivers capable of engaging in wireless, Radio Frequency (RF) communication. The transceiver(s) 1916 can also include other wireless modems, such as a modem for engaging in Wi-Fi, WiMAX, Bluetooth, or infrared communication.

In some implementations, the transceiver(s) 1916 can be used to communicate between various functions, components, modules, or the like, that are comprised in the device(s) 1900. For instance, the transceivers 1916 may facilitate communications between the vision screening device 104 and other devices storing the vision test 108, the code 112, the key 202, or the result 206.

### EXAMPLE CLAUSES

In some instances, one or more components may be referred to herein as “configured to,” “configurable to,” “operable/operative to,” “adapted/adaptable,” “able to,” “conformable/conformed to,” etc. Those skilled in the art will recognize that such terms (e.g., “configured to”) can generally encompass active-state components and/or inactive-state components and/or standby-state components, unless context requires otherwise.

As used herein, the term “based on” can be used synonymously with “based, at least in part, on” and “based at least partly on.”

As used herein, the terms “comprises/comprising/comprised” and “includes/including/included,” and their equivalents, can be used interchangeably. An apparatus, system, or method that “comprises A, B, and C” includes A, B, and C, but also can include other components (e.g., D) as well. That is, the apparatus, system, or method is not limited to components A, B, and C.

Although the subject matter has been described in language specific to structural features and/or methodological acts, it is to be understood that the subject matter defined in the appended claims is not necessarily limited to the specific features or acts described.

A: A vision screening system, comprising: an external medium displaying a vision test and a code; at least one camera configured to capture an image of the external medium; at least one input device configured to detect, from a subject, a response of the subject to viewing to the vision test; and a processor configured to: identify the code based on the image of the external medium; identify the vision test based on the code; determine based on the vision test and the response, whether an eye of the subject is characterized by a condition; and generate an output indicating whether the eye is characterized by the condition.

B: The vision screening system of paragraph A, wherein the image is a first image, the condition is a first condition, and the system further comprises a light source configured to project infrared radiation onto the eye of the subject, the camera being further configured to capture a second image of the eye, the second image being indicative of a response of the eye to the infrared radiation; and the processor being further configured to: determine, based on the second image, whether the eye is characterized by a second condition; and generate an additional output indicating whether the eye is characterized by the second condition.

C: The vision screening system of paragraph B, further comprising a transceiver, wherein the processor is configured to at least one of: cause the transceiver to provide a first signal, via a network, to an electronic device indicating whether the eye is characterized by the first condition; or cause the transceiver to provide a second signal, via the network, to the electronic device indicating whether the eye is characterized by the second condition.

D: The vision screening system of paragraph A, B, or C, wherein the external medium comprises at least one of: a printed substrate; a projector configured to project the vision test and the code; or a screen configured to display the vision test and the code.

E: The vision screening system of paragraph A, B, C, or D, wherein the vision test comprises at least one of: a color vision test; a reading comprehension test; a concussion test; a near vision test; a reading speed test; or a visual acuity test.

F: The vision screening system of paragraph A, B, C, D, or E, wherein the at least one input device comprises at least one of: a microphone configured to detect an audible signal indicative of the response; a touch sensor configured to detect a touch signal indicative of the response; or a button configured to receive a press signal indicative of the response.

G: The vision screening system of paragraph A, B, C, D, E, or F, wherein the image is a first image, and the at least one camera is configured to capture a second image of the eye, the second image being indicative of the response.

H: The vision screening system of paragraph A, B, C, D, E, F, or G, wherein: the at least one camera, the at least one input device, and the processor are integrated into a handheld housing, and the external medium is separate from the housing.

I: A method, comprising: capturing an image of an external medium; identifying a vision test associated with the external medium based on the image; receiving feedback characterizing the vision test from a subject; and determining whether the subject has an ocular condition based on the feedback characterizing the vision test.

J: The method of paragraph I, wherein identifying the vision test associated with the external medium based on the image comprises: identifying a code displayed by the external medium based on the image; and identifying the vision test based on the code.

K: The method of paragraph I or J, further comprising: receiving, from the external medium, at least one of an RFID signal or an NFC signal identifying the vision test.

L: The method of paragraph I, J, or K, wherein receiving feedback characterizing the vision test from the subject comprises receiving at least one of: a signal indicative of the subject tracing a shape on a substrate; an audio signal; or a signal indicative of the subject selecting an item on a substrate.

M: The method of paragraph I, J, K, or L, wherein determining whether the subject has the ocular condition comprises: identifying a key associated with the vision test; determining one or more discrepancies between the key and the feedback; and determining that the subject has the ocular condition based on the discrepancy.

N: The method of paragraph I, J, K, L, or M, further comprising: transmitting, to an external device, a signal indicating whether the subject is suspected to have the ocular condition; and storing the determination of whether the subject has the ocular condition.

O: The method of paragraph I, J, K, L, M, or N, further comprising outputting a signal indicating whether the subject is suspected to have the ocular condition.

P: A device, comprising: a processor; and memory storing instructions that, when executed by the processor, cause the processor to perform operations comprising: receiving a first signal from an external medium; identifying a vision test associated with the external medium based on the first signal; receiving a second signal from an input device, the second signal indicating a response of a subject viewing to the vision test; and determining, based on the vision test and the second signal, whether the subject has an ocular condition.

Q: The device of paragraph P, further comprising: at least one camera configured to capture an image of the external medium, wherein identifying the vision test associated with the external medium based on the first signal comprises: identifying a code displayed by the external medium based on the image; and identifying the vision test based on the code.

R: The device of paragraph P or Q, further comprising: a transceiver configured to receive the second signal from the external medium, the second signal comprising at least one of an RFID signal or an NFC signal.

S: The device of paragraph P, Q, or R, further comprising: one or more touch sensors configured to detect an indication of the subject touching the external medium, wherein the second signal includes a shape traced by the subject touching the external medium.

T: The device of paragraph P, Q, R, or S, wherein determining whether the subject has the ocular condition comprises: identifying a key associated with the vision test; determining one or more discrepancies between the key and the second signal; and determining that the subject has the ocular condition based on the one or more discrepancies between the key and the second signal.

What is claimed is:

1. A vision screening system, comprising:
an external medium displaying a vision test and a code;
at least one camera configured to capture an image of the external medium;
at least one input device configured to detect, from a subject, a response of the subject to viewing to the vision test; and
a processor configured to:
identify the code based on the image of the external medium;
identify the vision test based on the code;
determine based on the vision test and the response, whether an eye of the subject is characterized by a condition; and
generate an output indicating whether the eye is characterized by the condition.

2. The vision screening system of claim 1, wherein the image is a first image, the condition is a first condition, and the system further comprises a light source configured to project infrared radiation onto the eye of the subject,
the camera being further configured to capture a second image of the eye, the second image being indicative of a response of the eye to the infrared radiation; and
the processor being further configured to:
determine, based on the second image, whether the eye is characterized by a second condition; and
generate an additional output indicating whether the eye is characterized by the second condition.

3. The vision screening system of claim 2, further comprising a transceiver, wherein the processor is configured to at least one of:
cause the transceiver to provide a first signal, via a network, to an electronic device indicating whether the eye is characterized by the first condition; or
cause the transceiver to provide a second signal, via the network, to the electronic device indicating whether the eye is characterized by the second condition.

4. The vision screening system of claim 1, wherein the external medium comprises at least one of:
a printed substrate;
a projector configured to project the vision test and the code; or
a screen configured to display the vision test and the code.

5. The vision screening system of claim 1, wherein the vision test comprises at least one of:
a color vision test;
a reading comprehension test;
a concussion test;
a near vision test;
a reading speed test; or
a visual acuity test.

6. The vision screening system of claim 1, wherein the at least one input device comprises at least one of:
a microphone configured to detect an audible signal indicative of the response;
a touch sensor configured to detect a touch signal indicative of the response; or
a button configured to receive a press signal indicative of the response.

7. The vision screening system of claim 1, wherein the image is a first image, and the at least one camera is configured to capture a second image of the eye, the second image being indicative of the response.

8. The vision screening system of claim 1, wherein:
the at least one camera, the at least one input device, and the processor are integrated into a handheld housing, and
the external medium is separate from the housing.

9. A method, comprising:
capturing an image of an external medium;
identifying a vision test associated with the external medium based on the image;
receiving feedback characterizing the vision test from a subject; and
determining whether the subject has an ocular condition based on the feedback characterizing the vision test.

10. The method of claim 9, wherein identifying the vision test associated with the external medium based on the image comprises:
identifying a code displayed by the external medium based on the image; and
identifying the vision test based on the code.

11. The method of claim 9, further comprising:
receiving, from the external medium, at least one of an RFID signal or an NFC signal identifying the vision test.

12. The method of claim 9, wherein receiving feedback characterizing the vision test from the subject comprises receiving at least one of:
a signal indicative of the subject tracing a shape on a substrate;
an audio signal; or
a signal indicative of the subject selecting an item on a substrate.

13. The method of claim 9, wherein determining whether the subject has the ocular condition comprises:
identifying a key associated with the vision test;
determining one or more discrepancies between the key and the feedback; and
determining that the subject has the ocular condition based on the discrepancy.

14. The method of claim 9, further comprising:
transmitting, to an external device, a signal indicating whether the subject is suspected to have the ocular condition; and
storing the determination of whether the subject has the ocular condition.

15. The method of claim 9, further comprising outputting a signal indicating whether the subject is suspected to have the ocular condition.

16. A device, comprising:
a processor; and
memory storing instructions that, when executed by the processor, cause the processor to perform operations comprising:
receiving a first signal from an external medium;
identifying a vision test associated with the external medium based on the first signal;
receiving a second signal from an input device, the second signal indicating a response of a subject viewing to the vision test; and
determining, based on the vision test and the second signal, whether the subject has an ocular condition.

17. The device of claim 16, further comprising:
at least one camera configured to capture an image of the external medium,
wherein identifying the vision test associated with the external medium based on the first signal comprises:
identifying a code displayed by the external medium based on the image; and
identifying the vision test based on the code.

18. The device of claim 16, further comprising:
a transceiver configured to receive the second signal from the external medium, the second signal comprising at least one of an RFID signal or an NFC signal.

19. The device of claim 16, further comprising:
one or more touch sensors configured to detect an indication of the subject touching the external medium, wherein the second signal includes a shape traced by the subject touching the external medium.

20. The device of claim 16, wherein determining whether the subject has the ocular condition comprises:
identifying a key associated with the vision test;
determining one or more discrepancies between the key and the second signal; and
determining that the subject has the ocular condition based on the one or more discrepancies between the key and the second signal.
